(19) United States
(12) Patent Application Publication
(10) Pub. No.: US 2013/0116144 A1
(43) Pub. Date: May 9, 2013

(54) METHYLATION MARKER FOR DIAGNOSIS OF CERVICAL CANCER

(71) Applicants: Sung Whan An, Daejeon (KR); Young Ho Moon, Daejeon (KR); Tae Jeong Oh, Daejeon (KR)

(72) Inventors: Sung Whan An, Daejeon (KR); Young Ho Moon, Daejeon (KR); Tae Jeong Oh, Daejeon (KR)

(73) Assignee: GENOMICTREE, INC., DaeJeon (KR)

(21) Appl. No.: 13/642,407

(22) PCT Filed: Apr. 22, 2011
(86) PCT No.: PCT/KR11/02942

§ 371 Date: Dec. 28, 2012

(30) Foreign Application Priority Data
Apr. 23, 2010 (KR) 10-2010-0037710

(51) Int. Cl. C12Q 1/68

(52) U.S. Cl. CPC C12Q 1/6886
USPC 506/9; 536/24.1; 435/6.11; 506/16

(57) ABSTRACT

The present invention relates to a cervical cancer-specific methylation marker for diagnosis of cervical cancer, and more particularly, to a cervical cancer-specific methylation marker, the CpG island region of which is methylated specifically in cervical cancer cells. The use of the kit and nucleic acid chip for diagnosis of cervical cancer of the present invention enables diagnosis of cervical cancer at an initial transformation stage, thus making it possible to diagnose cervical cancer at an early stage. In addition, the invention makes it possible to predict progression for high-risk groups and screen cervical cancer in a more accurate and rapid manner compared to conventional methods.

### TECHNICAL FIELD

The present invention relates to a cervical cancer-specific methylation marker for diagnosis of cervical cancer, and more particularly, to a cervical cancer-specific biomarker, the CpG island region of which is methylated specifically in cervical cancer cells.

### BACKGROUND ART

Despite the current developed state of medical science, five-year survival rate of human cancers, particularly solid cancers (cancers other than blood cancer) that account for a large majority of human cancers, are less than 50%. About two-thirds of all cancer patients are detected at a progressed stage, and most of them die within two years after the diagnosis of cancer. Such poor results in cancer diagnosis and therapy are due not only to the problem of therapeutic methods, but also to the fact that it is not easy to diagnose cancer at an early stage or to diagnose progressed cancer accurately or to observe prognosis of the cancer for newly developed remedy.

In current clinical practice, the diagnosis of cancer typically is confirmed by performing tissue biopsy after history taking, physical examination and clinical assessment. However, the diagnosis of cancer by the existing clinical practices is possible only when the number of cancer cells is more than a billion, and the diameter of cancer is more than 1 cm. In this case, the cancer cells already have metastatic ability, and at least half thereof have already metastasized. Meanwhile, tumor markers for monitoring substances that are directly or indirectly produced from cancers, are used in cancer screening, but they cause confusion due to limitations in accuracy, since more than about half thereof appear normal even in the presence of cancer, and they often appear positive even in the absence of cancer. Furthermore, the anticancer agents that are mainly used in cancer therapy have a problem that they show an effect only when the volume of cancer is small.

The reason why the diagnosis and the treatment of cancer are difficult is that cancer cells are highly complex and variable. Cancer cells grow excessively and continuously, invading surrounding tissue and metastasize to distal organs leading to death. Despite the attack of an immune mechanism or anticancer therapy, cancer cells survive, continually develop, and cell groups that are most suitable for survival selectively propagate. Cancer cells are living bodies with a high degree of viability, which occur by the mutation of a large number of genes. In order that one cell is converted to a cancer cell and developed to a malignant cancer lump that is detectable in clinics, the mutation of a large number of genes must occur. Thus, in order to diagnose and treat cancer at the root, approaches at a gene level are necessary.

Recently, genetic analysis has been actively attempted to diagnose cancer. The simplest typical method is to detect the presence of ABL: BCR fusion genes (the genetic characteristic of leukemia) in blood by PCR. The method has an accuracy rate of more than 95%, and this simple and easy genetic analysis is being used for the therapy of chronic myelogenous leukemia, the evaluation of the therapeutic result and follow-up study. However, this method has a deficiency that it can be applied only to a few blood cancers.

Recently, genetic testing that uses a DNA in serum or blood plasma has been actively attempted. This is a method of detecting a cancer-related gene that is isolated from cancer cells and released into blood and present in the form of a free DNA in serum.

It is found that the concentration of DNA in serum is higher at cancer patients than that of normal persons by a factor of 5-10 times, and such increased DNA is released mostly from cancer cells. The analysis of cancer-specific gene abnormalities, such as the mutation, deletion and functional loss of oncogenes and tumor-suppressor genes, using such DNAs isolated from cancer cells, allows the diagnosis of cancer.

In this effort, there have been active attempts to diagnose lung cancer, head and neck cancer, breast cancer, colon cancer, and liver cancer by examining the promoter methylation of mutated K-Ras oncogenes, p53 tumor-suppressor genes and p16 genes in serum, and the labeling and instability of microsatellite (Chen, X. et al., Clin. Cancer Res., 5:2297, 1999; Esteller, M. et al., Cancer Res., 59:67, 1999; Sanchez-Cespedes. M. et al., Cancer Res., 60:892, 2000; Sozzi, G. et al., Clin. Cancer Res., 5:2689, 1999).

In samples other than blood, the DNA of cancer cells can also be detected. A method has been attempted in which the presence of cancer cells or oncogenes in sputum or bronchoalveolar lavage of lung cancer patients is detected by a gene or antibody test (Palmisano, W. et al., Cancer Res., 60:5954, 2000; Sueoka, E. et al., Cancer Res., 59:1404, 1999).

Additionally, other methods of detecting the presence of oncogenes in feces of colon and rectal cancer patients (Ahlquist, D. A. et al., Gastroenterol., 119:1219-27, 2000) and detecting promoter methylation abnormalities in urine and prostate fluid (Goessl, C. et al., Cancer Res., 60:5941, 2000) have been attempted. However, in order to accurately diagnose cancers that cause a large number of gene abnormalities and show various mutations characteristic of each cancer, a method, by which a large number of genes are simultaneously analyzed in an accurate and automatic manner, is required. However, such a method is not yet established.

Accordingly, methods of diagnosing cancer by the measurement of DNA methylation are being proposed. When the promoter CpG island of a certain gene is hyper-methylated, the expression of such a gene is silenced. This is interpreted to be a main mechanism by which the function of this gene is lost even when there is no mutation in the protein-coding sequence of the gene in a living body. Also, this is analyzed as a factor by which the function of a number of tumor-suppressor genes in human cancer is lost. Thus, detecting the methylation of the promoter CpG island of tumor-suppressor genes is greatly needed for the study of cancer. Recently, an attempt has actively been conducted to determine promoter methylation, by methods such as methylation-specific PCR (hereinafter referred to as “MSP”) or automatic DNA sequencing, for diagnosis and screening of cancer.

A significant number of diseases are caused by genetic abnormalities, and the most frequent forms of genetic abnormalities are changes in gene-coding sequences. Such genetic changes are called mutations. When any gene has a mutation, the structure and function of a protein encoded by the gene change, resulting in abnormalities and deletion, and this mutant protein causes a disease. However, an abnormality in the expression of a specific gene can cause disease even in the absence of a mutation in the gene. A typical example thereof is methylation in which a methyl group is attached to the transcription regulatory region of a gene, that is, the cytosine base of the promoter CpG islands, in this case, the expression of this gene is silenced. This is known as an epigenetic change. This is transmitted to offspring and results in the loss of the expression of the relevant protein in the same manner as mutation. Most typically, the expression of tumor suppressor genes is silenced by the methylation of promoter CpG islands in cancer cells, resulting in carcinogenesis (Robertson, K. D. et al., Carcinogensis, 21:461, 2000).

For the accurate diagnosis of cancer, it is important not only to detect a mutated gene but also to decide the site where the mutation of this gene occurs. Previously, studies were conducted focusing on mutations in a coding sequence, i.e., micro-changes, such as point mutations, deletions and insertions, or macroscopic chromosomal abnormalities. However, in recent years, epigenetic changes were reported to be as important as these mutations, and a typical example of the epigenetic changes is the methylation of promoter CpG islands.

In the genomic DNA of mammal cells, there is the fifth base in addition to A, C, G and T, namely, 5-methylcytosine, in which a methyl group is attached to the fifth carbon of the cytosine ring (5-mC). 5-mC is always attached only to the C of a CG dinucleotide (5′-mCG-3′), which is frequently marked CpG. The C of CpG is mostly methylated by attachment with a methyl group. The methylation of this CpG inhibits a repetitive sequence in genomes, such as Alu or transposon, from being expressed. In addition, this CpG is a site where an epigenetic change in mammalian cells appears most often. The 5-mC of this CpG is naturally deaminated to T, and thus, the CpG in mammal genomes shows only 1% of frequency, which is much lower than a normal frequency (¼×¼=6.25%).

Regions in which CpG are exceptionally integrated are known as CpG islands. The CpG islands refer to sites which are 0.2-3 kb in length, and have a C+G content of more than 50% and a CpG ratio of more than 3.75%. There are about 45,000 CpG islands in the human genome, and they are mostly found in promoter regions regulating the expression of genes. Actually, the CpG islands occur in the promoters of housekeeping genes accounting for about 50% of human genes (Cross, S. et al., Curr. Opin. Gene Develop., 5:309, 1995).

In the meantime, in the somatic cells of normal persons, the CpG islands of such housekeeping gene promoter sites are un-methylated, but imprinted genes and the genes on inactivated X chromosomes are methylated such that they are not expressed during development.

During a cancer-causing process, methylation is found in promoter CpG islands, and the restriction on the corresponding gene expression occurs. Particularly, if methylation occurs in the promoter CpG islands of tumor-suppressor genes that regulate cell cycle or apoptosis, restore DNA, are involved in the adhesion of cells and the interaction between cells, and/or suppress cell invasion and metastasis, such methylation blocks the expression and function of such genes in the same manner as the mutations of a coding sequence, thereby promoting the development and progression of cancer. In addition, partial methylation also occurs in the CpG islands according to aging.

An interesting fact is that, in the case of genes whose mutations are attributed to the development of cancer in congenital cancer but do not occur in acquired cancer, the methylation of promoter CpG islands occurs instead of mutation. Typical examples include acquired renal cancer VHL (von Hippel Lindau), breast cancer BRCA1, colon cancer MLH1, and stomach cancer E-CAD. In addition, in about half of all cancers, the promoter methylation of p16 or the mutation of Rb occurs, and the remaining cancers show the mutation of p53 or the promoter methylation of p73, p 14 and the like.

An important fact is that an epigenetic change caused by promoter methylation causes a genetic change (i.e., the mutation of a coding sequence), and the development of cancer is progressed by the combination of such genetic and epigenetic changes. In a MLH1 gene as an example, there is the circumstance in which the function of one allele of the MLH1 gene in colon cancer cells is lost due to its mutation or deletion, and the remaining one allele does not function due to promoter methylation. In addition, if the function of MLH1, which is a DNA restoring gene, is lost due to promoter methylation, the occurrence of mutation in other important genes is facilitated to promote the development of cancer.

Most cancers show three common characteristics with respect to CpG, namely, hypermethylation of the promoter CpG islands of tumor-suppressor genes, hypermethylation of the remaining CpG base sites, and an increase in the activity of methylation enzyme, namely, DNA cytosine methyltransferase (DNMT) (Singal, R. & Ginder, G. D., Blood, 93:4059, 1999; Robertson, K. et al., Carcinogensis, 21:461, 2000; Malik, K. & Brown, K. W., Brit. J. Cancer, 83:1583, 2000).

When promoter CpG islands are methylated, the reason why the expression of the corresponding genes is blocked is not clearly established, but is presumed to be because a methyl CpG-binding protein (MECP) or a methyl CpG-binding domain protein (MBD), and histone deacetylase, bind to methylated cytosine, thereby causing a change in the chromatin structure of chromosomes and a change in histone protein.

It is unsettled whether the methylation of promoter CpG islands directly causes the development of cancer or is a secondary change after the development of cancer. However, it is clear that the promoter methylation of tumor-suppressor genes is an important index to cancer, and thus can be used in many applications, including the diagnosis and early detection of cancer, the prediction of the risk of the development of cancer, the prognosis of cancer, follow-up examination after treatment, and the prediction of a response to anticancer therapy. Recently, an attempt to examine the promoter methylation of tumor-suppressor genes in blood, sputum, saliva, feces or urine and to use the examined results for the diagnosis and treatment of various cancers, has been actively conducted (Esteller, M. et al., Cancer Res., 59:67, 1999; Sanchez-Cespedez, M. et al., Cancer Res., 60:892, 2000; Ahlquist, D. A. et al., Gastroenterol., 119:1219, 2000).

In order to maximize the accuracy of cancer diagnosis using promoter methylation, analyze the development of cancer according to each stage and discriminate a change according to cancer and aging, an examination that can accurately analyze the methylation of all the cytosine bases of promoter CpG islands is required. Currently, a standard method for this examination is a bisulfite genome-sequencing method, in which a sample DNA is treated with sodium bisulfite, and all regions of the CpG islands of a target gene to be examined is amplified by PCR, and then, the base sequence of the amplified regions is analyzed. However, this examination has the problem that there are limitations to the number of genes or samples that can be examined at a given time. In addition, such examination entails another problem in that automation is difficult, and much time and expense are required.

The methylation of promoter CpG islands has a deep connection with physiological phenomena, such as the development and differentiation of the human body, and also aging, the development of various cancers and diseases. Particularly, the methylation of the promoter CpG islands of tumor-related genes can act as an index of cancer since they play an important role in the development and progression of cancer. In particular, in cervical cancer, for instance, stages of cancer progression have been categorized, such as SIL (squamous intraepithelial lesion), which indicates dysplasia generally; LSIL (low squamous intraepithelial lesion), which indicates mild dysplasia; HSIL (high squamous intraepithelial lesion), which indicates moderate to severe dysplasia; CIS (carcinoma in situ); and Squamous cell carcinoma. Accordingly, it is desirable to find marker genes that are specific for these stages of cancer progression.

Conventional methods utilize amplification of regions of genes containing CpG islands by methylation specific PCR (MSP) together with a base sequence analysis method (bisulfite genome-sequencing method). However, there is no method that can analyze various changes of the promoter methylation of many genes at a given time in an accurate, rapid and automatic manner, and can be applied to the diagnosis, early diagnosis or assessment of each stage of various cancers in clinical practice.

Accordingly, the present inventors have made extensive efforts to develop a diagnostic kit capable of effectively diagnosing cervical cancer and, as a result, have found that cervical cancer can be diagnosed by measuring the degree of methylation using the promoter of a methylation-related gene, which is methylated specifically in cervical cancer cells, as a biomarker, thereby completing the present invention.

### DISCLOSURE OF INVENTION

It is a main object of the present invention to provide a composition for diagnosis of cervical cancer and a kit and chip for diagnosing cervical cancer at an early stage using the same.

To achieve the above object, the present invention provides a composition for diagnosis of cervical cancer, which contains the promoter CpG island or the UTR CpG island of a cervical cancer-specific biomarker ACSS3 (NM_{—}024506, acyl-CoA synthetase short-chain family member 3).

The present invention also provides a kit for diagnosis of cervical cancer, which comprises: a PCR primer pair for amplifying a fragment comprising the promoter CpG island of ACSS3 gene or the UTR CpG island of the ACSS3 gene; and a sequencing primer for sequencing a PCR product amplified by the primer pair.

The present invention also provides a nucleic acid chip for diagnosis of cervical cancer, which comprises a probe capable of hybridizing with a fragment, comprising the promoter CpG island of ACSS3 gene or the UTR CpG island of the ACSS3 gene, under strict conditions.

The present invention also provides a method of detecting methylation of the above biomarker from a clinical sample containing DNA.

Other features and embodiments of the present invention will be more apparent from the following detailed descriptions and the appended claims

### BRIEF DESCRIPTION OF THE DRAWINGS

FIG. 1 is a schematic diagram showing a process of discovering a methylation biomarker for diagnosis of cervical cancer from the scrapes of normal persons and cervical patients by CpG microarray analysis.

FIG. 2 shows a process of selecting cervical cancer-specific methylation biomarker ACSS3 gene using the method shown in FIG. 1.

FIG. 3 shows the results of measuring the degree of methylation of the promoter region of ACSS3 methylation biomarker gene in cervical cancer cell lines by a pyrosequencing method.

FIG. 4 shows the results of measuring the methylation degree of the promoter region of ACSS3 methylation biomarker gene in cervical cancer scrapes by a pyrosequencing method and also shows an ROC curve for diagnosis of cervical cancer.

FIG. 5 shows the results of measuring the methylation degree of the promoter region of ADCYAP1 biomarker gene in cervical cancer scrapes by a pyrosequencing method and also shows an ROC curve for diagnosis of cervical cancer.

FIG. 6 shows the results of measuring the methylation degree of the promoter region of ADCYAP1 biomarker gene in cervical cancer scrapes by a pyrosequencing method and also shows an ROC curve for diagnosis of cervical cancer.

FIG. 7 shows the results of measuring the methylation degree of the promoter region of HOXA11 biomarker gene in cervical cancer scrapes by a pyrosequencing method and also shows an ROC curve for diagnosis of cervical cancer.

FIG. 8 shows the results of measuring the methylation degree of the promoter region of VIM biomarker gene in cervical cancer scrapes by a pyrosequencing method and also shows an ROC curve for diagnosis of cervical cancer.

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein are well known and are commonly employed in the art.

The definition of main terms used in the detailed description of the invention is as follows.

As used herein, the term “cell transformation” refers to the change in characteristics of a cell from one form to another form such as from normal to abnormal, non-tumorous to tumorous, undifferentiated to differentiated, stem cell to non-stem cell. In addition, the transformation can be recognized by the morphology, phenotype, biochemical characteristics and the like of a cell.

As used herein, the term “early detection” of cancer refers to discovering the likelihood of cancer prior to metastasis, and preferably before observation of a morphological change in a tissue or cell. Furthermore, the term “early detection” of cell transformation refers to the high probability of a cell to undergo transformation in its early stages before the cell is morphologically designated as being transformed.

As used herein, the term “hypermethylation” refers to the methylation of a CpG island.

As used herein, the term “sample” or “clinical sample” is referred to in its broadest sense, and includes any biological sample obtained from an individual, body fluid, a cell line, a tissue culture, depending on the type of assay that is to be performed. Methods for obtaining tissue biopsies and body fluids from mammals are well known in the art. A tissue biopsy of the cervix is a preferred source.

As used herein, “transformed” cell refers to the change in characteristics of a cell or tissue from one form to another such as from normal to abnormal, non-tumorous to tumorous, undifferentiated to differentiated, and so on.

In one aspect, the present invention is directed to a composition for diagnosis of cervical cancer, which contains the promoter CpG island of a cervical cancer-specific biomarker ACSS3 (NM_{—}024506, acyl-CoA synthetase short-chain family member 3) gene or the UTR CpG island of the gene.

In the present invention, the promoter region of the gene may contain at least one methylated CpG dinucleotide. Herein, the promoter or UTR region of the ACSS3 gene may comprise a nucleotide sequence of SEQ ID NO: 1 or 2 which corresponds nucleotides −500 to +90 nt with respect to the transcription start site (+1) of the gene.

The present invention may be directed to a composition for diagnosis of cervical cancer, which further contains the methylated CpG island of one selected from the group consisting of the promoter or UTR of cervical cancer-specific biomarker ADCYAP1 gene, the promoter of HOXA11 gene and the promoter of VIM gene. Herein, the promoter or UTR region of ADCYAP1 gene may comprise a nucleotide sequence of SEQ ID NO: 6 or 7 which corresponds to nucleotides −500 to +500 with respect to the transcription start site of the gene, and the promoter region of HOXA11 gene may comprise a nucleotide sequence of SEQ ID NO: 14 which corresponds to nucleotides −800 to −1 with respect to the transcription start site of the gene. In addition, the promoter region of VIM gene may comprise a nucleotide sequence of SEQ ID NO: 18 which corresponds to nucleotides −1200 to −500 with respect to the transcription start site of the gene.

In one embodiment of the present invention, nucleic acid can be methylated in the regulatory region of a gene. In another embodiment, methylation begins from the outer boundary of the regulatory region of a gene and then spreads inward, and thus detection of methylation at the outer boundary of the regulatory region enables early diagnosis of genes which are involved in cell transformation.

In still another embodiment of the present invention, the cell growth abnormality (dysplasia) of cervical tissue in a sample can be diagnosed by detecting the methylation of at least one of the following nucleic acids using a kit: the promoter of ACSS3 gene, and a combination of the promoter of ACSS3 gene and any one of the promoter or UTR of ADCYAP1 gene, the promoter of HOXA11 gene, and the promoter of VIM gene.

The method of the present invention enables diagnosis of cervical cancer progression at various stages by determining the methylation stage of at least one nucleic acid biomarker obtained from a sample. When the methylation stage of nucleic acid isolated from a sample at each stage of cervical cancer is compared with the methylation stage of at least one nucleic acid obtained from a sample having no abnormality in the cell proliferation of cervical tissue, a certain stage of cervical cancer in the sample can be determined. The methylation stage may be hypermethylation.

The use of the diagnostic kit of the present invention can determine the cell growth abnormality (dysplasia progression) of cervical tissue in a sample. The method for determining the cell growth abnormality of cervical tissue comprises determining the methylation of at least one nucleic acid isolated from a sample. In the method, the methylation stage of at least one nucleic acid is compared with the methylation stage of a nucleic acid isolated from a sample having no cell growth abnormality (dysplasia).

Examples of the nucleic acid include the promoter or UTR of ACSS3 gene, and a combination of the promoter or UTR of ACSS3 gene and any one of the promoter or UTR of ADCYAP1 gene, the promoter of HOXA11, and the promoter of VIM gene.

Also, the methylation state of the CpG island can be determined by a kit for diagnosis of cervical cancer, which contains: a PCR primer pair for amplifying a fragment comprising the CpG island of each gene region; and a sequencing primer for sequencing a PCR product amplified by the primer pair.

In the present invention, the PCR primer pair may be a combination of a primer pair of SEQ ID NOS: 3 and 4 and a primer pair selected from the group consisting of a primer pair of SEQ ID NOS: 8 and 9, a primer pair of SEQ ID NOS: 11 and 12, a primer pair of SEQ ID NOS: 15 and 16, and a primer pair of SEQ ID NOS: 19 and 20.

In the present invention, the sequencing primer may be a combination of a sequencing primer of SEQ ID NO: 5 and a sequencing primer selected from the group consisting of a sequencing primer of SEQ ID NO: 5, a sequencing primer of SEQ ID NO: 10, a sequencing primer of SEQ ID NO: 13, a sequencing primer of SEQ ID NO: 17, and a sequencing primer of SEQ ID NO: 21.

In still another embodiment of the present invention, a cell capable of forming cervical cancer can be diagnosed at an early stage using the methylation gene marker. When a gene confirmed to be methylated in a cancer cell is methylated in a cell which appears to be normal clinically or morphologically, carcinogenesis of the cell that appears to be normal is determined to be in progress. Thus, cervical cancer can be diagnosed at an early stage by detecting the methylation of a cervical cancer-specific gene in a cell that appears to be normal.

The use of the methylation marker gene of the present invention enables detection of the cell growth abnormality (dysplasia progression) of cervical tissue in a sample. The method for detecting the cell growth abnormality of cervical tissue comprises bringing at least one nucleic acid isolated from a sample into contact with an agent capable of determining the methylation status of the nucleic acid. The method comprises determining the methylation status of at least one region in at least one nucleic acid, and the methylation status of the nucleic acid differs from the methylation status of the same region in a nucleic acid isolated from a sample having no cell growth abnormality (dysplasia progression) of cervical tissue.

In still another embodiment of the present invention, a transformed cervical cancer cell can be detected by examining the methylation of a marker gene using the above-described kit.

In still another embodiment of the present invention, the likelihood of progression to cervical cancer can be diagnosed by examining the methylation of a marker gene with the above-described kit in a sample showing a normal phenotype. The sample may be solid or liquid tissue, cell, urine, serum or plasma.

In another aspect, the present invention is directed to a nucleic acid chip for diagnosis of cervical cancer, which comprises a probe capable of hybridizing with a fragment, comprising the promoter CpG island or the UTR CpG island of ACSS3 gene, under strict conditions.

In the present invention, the nucleotide chip may further comprise a probe capable of hybridizing with a fragment comprising the CpG island of one selected from the group consisting of the promoter or UTR of ADCYAP1 gene, the promoter of HOXA11 gene, and the promoter of VIM gene, under strict conditions.

In still another aspect, the present invention is also directed to a method of detecting methylation of the above biomarker from a clinical sample containing DNA.

In the present invention, the methylation detection method may be performed using a method selected from the group consisting of PCR, methylation-specific PCR, real-time methylation-specific PCR, PCR using methylated DNA-specific binding protein, quantitative PCR, pyrosequencing, and bisulfite sequencing. In addition, the clinical sample may comprise a tissue, cell, blood or urine derived from a diagnosed subject or a cancer-suspected patient.

The inventive method for detecting promoter methylation of a gene comprises the steps of: (a) separating sample DNA from a clinical sample; and (b) detecting methylation of the promoter CpG island of ACSS3 gene from the DNA of the clinical sample. In step (b), whether the promoter was methylated can be determined by amplifying the isolated DNA using primers, which can amplify a CpG island-containing fragment comprising the ACSS3 gene promoter or a combination of the ACSS3 gene promoter and any one of the ADCYAP1 gene promoter or UTR, the HOXA11 gene promoter and VIM gene promoter, and determining whether an amplification product was obtained from the DNA or whether the nucleotide sequence of the DNA was changed.

In yet another embodiment of the present invention, the likelihood of development of tissue to cervical cancer can be evaluated by examining the methylation frequency of a gene which is methylated specifically in cervical cancer and determining the methylation frequency of tissue having the likelihood of progression to cervical cancer.

Screening of Methylation-Regulated Biomarker

The present invention is directed to a method of determining a biomarker gene which is methylated when a cell or tissue is transformed or changed from one type of cell to another. As used herein, “transformed” cell refers to the change in characteristics of a cell or tissue from one form to another such as from normal to abnormal, non-tumorous to tumorous, undifferentiated to differentiated and so on.

In one Example of the present invention, genomic DNAs were isolated from the scrapes of normal persons and cervical cancer patients. In order to obtain only methylated DNAs from the genomic DNAs, the genomic DNAs were allowed to react with MBD2bt binding to methylated DNA, and then methylated DNAs binding to the MBD2bt protein were isolated. The isolated methylated DNAs binding to the McrBt protein were amplified, and then the DNAs originated from the normal persons were labeled with Cy3, and the DNAs originated from the cervical cancer patients were labeled with Cy5. Then, the DNAs were hybridized to human CpG-island microarrays, and ACSS3 gene showing the greatest difference in methylation degree between the normal persons and the cervical cancer patients were selected as a biomarker.

In the present invention, in order to further confirm whether the biomarker has been methylated, pyrosequencing was performed.

Specifically, total genomic DNA was isolated from the cervical cell lines C33A, HeLa, SiHa and Caski and treated with bisulfite. The genomic DNA converted with bisulfite was amplified. Then, the amplified PCR product was subjected to pyrosequencing in order to measure the methylation degree of the gene. As a result, it could be seen that the biomarker was methylated.

Biomarker for Cervical Cancer

The present invention provides a biomarker for diagnosing cervical cancer.

Biomarker for Cervical Cancer—Use of Cancer Cells for Comparison with Normal Cells

In one aspect, the present invention is based on the discovery of the relationship between cervical cancer and the hypermethylation of the promoter or UTR region of the following four genes: ACSS3 (NM_{—}024506, acyl-CoA synthetase short-chain family member 3) gene and combinations of ACSS3 gene with ADCYAP1 (NM_{—}001099733, adenylate cyclase activating polypeptide 1, pituitary), HOXA11 (NM_{—}005523, homeobox A11) and VIM (NM_{—}003380, Vimentin) genes.

In another embodiment of the present invention, a cellular proliferative disorder of cervical tissue cell of a subject can be diagnosed at an early stage by determining the methylation stage of at least one nucleic acid isolated from the subject using the kit of the present invention. Herein, the methylation stage of the at least one nucleic acid may be compared with the methylation state of at least one nucleic acid isolated from a subject not having a cellular proliferative disorder of cervical tissue. The nucleic acid is preferably a CpG-containing nucleic acid such as a CpG island.

In other applications of the diagnostic kit of the present invention, the cell growth abnormality of cervical tissue in a subject can be diagnosed by a method comprising determining the methylation of one or more nucleic acids isolated from the subject. The nucleic acids are preferably those encoding ACSS3 (NM_{—}024506, acyl-CoA synthetase short-chain family member 3) genes and combinations of ACSS3 gene with ADCYAP1 (NM_{—}001099733, adenylate cyclase activating polypeptide 1, pituitary), HOXA11 (NM_{—}005523, homeobox A11) and VIM (NM_{—}003380, Vimentin) genes. In this embodiment, the methylation of the at least one nucleic acid may be compared with the methylation state of at least one nucleic acid isolated from a subject having no predisposition to a cellular proliferative disorder of the cervical tissue.

As used herein, the term “predisposition” refers to the property of being susceptible to a cellular proliferative disorder. A subject having a predisposition to a cellular proliferative disorder has no cellular proliferative disorder, but is a subject having an increased likelihood of having a cellular proliferative disorder.

In another aspect, the present invention provides a method for diagnosing a cellular proliferative disorder of a cervical tissue, the method comprising bringing a sample comprising a nucleic acid into contact with an agent capable of determining the methylation state of the sample, and determining the methylation of at least one region of the at least one nucleic acid. Herein, the methylation of the at least one region in the at least one nucleic acid differs from the methylation stage of the same region in a nucleic acid present in a subject in which there is no abnormal growth of cells.

The method of the present invention comprises a step of determining the methylation of at least one region of at least one nucleic acid isolated from a subject.

The term “nucleic acid” or “nucleic acid sequence” as used herein refers to an oligonucleotide, nucleotide or polynucleotide, or fragments thereof, or single-stranded or double-stranded DNA or RNA of genomic or synthetic origin, sense- or antisense-strand DNA or RNA of genomic or synthetic origin, peptide nucleic acid (PNA), or any DNA-like or RNA-like material of natural or synthetic origin. It will apparent to those of skill in the art that, when the nucleic acid is RNA, the deoxynucleotides A, G, C, and T are replaced by the ribonucleotides A, G, C, and U, respectively.

Any nucleic acid may be used in the present invention, as long as the presence of differently methylated CpG islands can be detected therein. The CpG island is a CpG-rich region in a nucleic acid sequence.

Methylation

In the present invention, any nucleic acid sample, in purified or nonpurified form, can be used, provided it contains or is suspected of containing a nucleic acid sequence containing a target locus (e.g., CpG-containing nucleic acid). One nucleic acid region capable of being differentially methylated is a CpG island, a sequence of nucleic acid with an increased density relative to other nucleic acid regions of the dinucleotide CpG. The CpG doublet occurs in vertebrate DNA at only about 20% of the frequency that would be expected from the proportion of G*C base pairs. CpG islands have an average G*C content of about 60%, compared with the 40% average in bulk DNA. The islands take the form of stretches of DNA typically about one to two kilobases long. There are about 45,000 islands in the human genome.

In many genes, the CpG islands begin just upstream of a promoter and extend downstream into the transcribed region. Methylation of a CpG island at a promoter usually suppresses expression of the gene. The islands can also surround the 5′ region of the coding region of the gene as well as the 3′ region of the coding region. Thus, CpG islands can be found in multiple regions of a nucleic acid sequence including upstream of coding sequences in a regulatory region including a promoter region, in the coding regions (e.g., exons), downstream of coding regions in, for example, enhancer regions, and in introns.

Typically, the CpG-containing nucleic acid is DNA. However, the inventive method may employ, for example, samples that contain DNA, or DNA and RNA containing mRNA, wherein DNA or RNA may be single-stranded or double-stranded, or a DNA-RNA hybrid may be included in the sample.

A mixture of nucleic acids may also be used. The specific nucleic acid sequence to be detected may be a fraction of a larger molecule or can be present initially as a discrete molecule, so that the specific sequence constitutes the entire nucleic acid. It is not necessary that the sequence to be studied be present initially in a pure form; the nucleic acid may be a minor fraction of a complex mixture, such as contained in whole human DNA. Nucleic acids contained in a sample used for detection of methylated CpG islands may be extracted by a variety of techniques such as that described by Sambrook, et al. (Molecular Cloning: A Laboratory Manual, Cold Spring Harbor, N.Y., 1989).

A nucleic acid can contain a regulatory region which is a region of DNA that encodes information or controls transcription of the nucleic acid. Regulatory regions include at least one promoter. A “promoter” is a minimal sequence sufficient to direct transcription, to render promoter-dependent gene expression controllable for cell-type specific, tissue-specific, or inducible by external signals or agents. Promoters may be located in the 5′ or 3′ region of the gene. Promoter regions, in whole or in part, of a number of nucleic acids can be examined for sites of CpG-island methylation. Moreover, it is generally recognized that methylation of the target gene promoter proceeds from the outer boundary inward. Therefore, the early stage of cell conversion can be detected by analyzing methylation in these outer areas of the promoter region.

Nucleic acids isolated from a subject are obtained in a biological sample from the subject. If it is desired to detect cervical cancer or stages of cervical cancer progression, the nucleic acid must be isolated from cervical tissue by scraping or biopsy. Such samples may be obtained by various medical procedures known to those of skill in the art.

In one aspect of the invention, the state of methylation in nucleic acids of the sample obtained from a subject is hypermethylation compared with the same regions of the nucleic acid in a subject not having a cellular proliferative disorder of cervical tissue. Hypermethylation as used herein refers to the presence of methylated alleles in one or more nucleic acids. Nucleic acids from a subject not having a cellular proliferative disorder of cervical tissue contain no detectable methylated alleles when the same nucleic acids are examined.

Sample

The present invention describes early diagnosis of cervical cancer and utilizes the methylation of cervical cancer-specific genes. The methylation of cervical cancer-specific genes also occurred in tissue near tumor sites. Therefore, in the method for early diagnosis of cervical cancer, the methylation of cervical cancer-specific genes can be detected by examining all samples, including liquid or solid tissue. The samples include, but are not limited to, tissue, cell, urine, serum or plasma.

Individual Genes and Panel

It is understood that the present invention may be practiced using ACSS3 gene separately as a diagnostic or prognostic marker or ADCYAP1, HOXA11 and VIM marker genes combined into a panel display format so that ACSS3 gene, ADCYAP1, HOXA11 and VIM marker genes may be detected for overall pattern or listing of genes that are methylated to increase reliability and efficiency. Furthermore, any of the genes identified in the present invention may be used individually or as a set of genes in any combination with any of the other genes that are recited herein. Alternatively, genes may be ranked according to their importance and weighted and together with the number of genes that are methylated, a level of likelihood of developing cancer may be assigned. Such algorithms are within the scope of the present invention.

Method for Detection of Methylation

Methylation-Specific PCR

When genomic DNA is treated with bisulfite, cytosine in the 5′-CpG′-3 region remains intact, if it was methylated, but the cytosine changes to uracil, if it was unmethylated. Accordingly, based on the base sequence converted after bisulfite treatment, PCR primer sets corresponding to a region having the 5′-CpG-3′ base sequence are constructed. Herein, the constructed primer sets are two kinds of primer sets: a primer set corresponding to the methylated base sequence, and a primer set corresponding to the unmethylated base sequence. When genomic DNA is converted with bisulfite and then amplified by PCR using the above two kinds of primer sets, the PCR product is detected in the PCR mixture employing the primers corresponding to the methylated base sequence, if the genomic DNA was methylated, but the genomic DNA is detected in the PCR mixture employing the primers corresponding to the unmethylated, if the genomic DNA was unmethylated. This methylation can be qualitatively analyzed by agarose gel electrophoresis.

Real-Time Methylation Specific PCR

Real-time methylation-specific PCR is a real-time measurement method modified from the methylation-specific PCR method and comprises treating genomic DNA with bisulfite, designing PCR primers corresponding to the methylated base sequence, and performing real-time PCR using the primers. Methods of detecting the methylation of the genomic DNA include two methods: a method of detection using a TanMan probe complementary to the amplified base sequence; and a method of detection using LCgreen. Thus, the real-time methylation-specific PCR allows selective quantitative analysis of methylated DNA. Herein, a standard curve is plotted using an in vitro methylated DNA sample, and a gene containing no 5′-CpG-3′ sequence in the base sequence is also amplified as a negative control group for standardization to quantitatively analyze the degree of methylation.

Pyrosequencing

The pyrosequencing method is a quantitative real-time sequencing method modified from the bisulfite sequencing method. Similarly to bisulfite sequencing, genomic DNA is converted by bisulfite treatment, and then, PCR primers corresponding to a region containing no 5′-CpG-3′ base sequence are constructed. Specifically, the genomic DNA is treated with bisulfite, amplified using the PCR primers, and then subjected to real-time base sequence analysis using a sequencing primer. The degree of methylation is expressed as a methylation index by analyzing the amounts of cytosine and thymine in the 5′-CpG-3′ region.

PCR Using Methylated DNA-Specific Binding Protein, Quantitative PCR, and DNA Chip Assay

When a protein binding specifically only to methylated DNA is mixed with DNA, the protein binds specifically only to the methylated DNA. Thus, either PCR using a methylation-specific binding protein or a DNA chip assay allows selective isolation of only methylated DNA. Genomic DNA is mixed with a methylation-specific binding protein, and then only methylated DNA was selectively isolated. The isolated DNA is amplified using PCR primers corresponding to the promoter region, and then methylation of the DNA is measured by agarose gel electrophoresis.

In addition, methylation of DNA can also be measured by a quantitative PCR method, and methylated DNA isolated with a methylated DNA-specific binding protein can be labeled with a fluorescent probe and hybridized to a DNA chip containing complementary probes, thereby measuring methylation of the DNA. Herein, the methylated DNA-specific binding protein may be, but not limited to, MBD2bt.

Detection of Differential Methylation—Methylation Sensitive Restriction Endonuclease

Detection of differential methylation can be accomplished by bringing a nucleic acid sample into contact with a methylation-sensitive restriction endonuclease that cleaves only unmethylated CpG sites.

In a separate reaction, the sample is further brought into contact with an isochizomer of the methylation-sensitive restriction enzyme that cleaves both methylated and unmethylated CpG-sites, thereby cleaving the methylated nucleic acid.

Specific primers are added to the nucleic acid sample, and the nucleic acid is amplified by any conventional method. The presence of an amplified product in the sample treated with the methylation-sensitive restriction enzyme but absence of an amplified product in the sample treated with the isochizomer of the methylation-sensitive restriction enzyme indicates that methylation has occurred at the nucleic acid region assayed. However, the absence of an amplified product in the sample treated with the methylation-sensitive restriction enzyme together with the absence of an amplified product in the sample treated with the isochizomer of the methylation-sensitive restriction enzyme indicates that no methylation has occurred at the nucleic acid region assayed.

As used herein, the term “methylation-sensitive restriction enzyme” refers to a restriction enzyme (e.g., SmaI) that includes CG as part of its recognition site and has activity when the C is methylated as compared to when the C is not methylated. Non-limiting examples of methylation-sensitive restriction enzymes include MspI, HpaII, BssHII, BstUI and NotI. Such enzymes can be used alone or in combination. Examples of other methylation-sensitive restriction enzymes include, but are not limited to SacII and EagI.

The isochizomer of the methylation-sensitive restriction enzyme is a restriction enzyme that recognizes the same recognition site as the methylation-sensitive restriction enzyme but cleaves both methylated and unmethylated CGs. An example thereof includes MspI.

Primers of the present invention are designed to be “substantially” complementary to each strand of the locus to be amplified and include the appropriate G or C nucleotides as discussed above. This means that the primers must be sufficiently complementary to hybridize with their respective strands under polymerization reaction conditions. Primers of the present invention are used in the amplification process, which is an enzymatic chain reaction (e.g., PCR) in which that a target locus exponentially increases through a number of reaction steps. Typically, one primer is homologous with the negative (−) strand of the locus (antisense primer), and the other primer is homologous with the positive (+) strand (sense primer). After the primers have been annealed to denatured nucleic acid, the nucleic acid chain is extended by an enzyme such as DNA Polymerase I (Klenow), and reactants such as nucleotides, and, as a result, + and − strands containing the target locus sequence are newly synthesized. When the newly synthesized target locus is used as a template and subjected to repeated cycles of denaturing, primer annealing, and extension, exponential synthesis of the target locus sequence occurs. The resulting reaction product is a discrete nucleic acid duplex with termini corresponding to the ends of specific primers employed.

Preferably, the amplification reaction is PCR which is commonly used in the art to which the present invention pertains. However, alternative methods such as real-time PCR or linear amplification using isothermal enzyme may also be used. In addition, multiplex amplification reactions may also be used.

Detection of Differential Methylation—Bisulfate Sequencing Method

Another method for detecting a methylated CpG-containing nucleic acid comprises the steps of: bringing a nucleic acid-containing sample into contact with an agent that modifies unmethylated cytosine; and amplifying the CpG-containing nucleic acid in the sample using CpG-specific oligonucleotide primers, wherein the oligonucleotide primers distinguish between modified methylated nucleic acid and non-methylated nucleic acid and detect the methylated nucleic acid. The amplification step is optional and desirable, but not essential. The method relies on the PCR reaction to distinguish between modified (e.g., chemically modified) methylated DNA and unmethylated DNA. Such methods are described in U.S. Pat. No. 5,786,146 relating to bisulfite sequencing for detection of methylated nucleic acid.

Substrates

After the target nucleic acid region has been amplified, the nucleic acid amplification product can be hybridized to a known gene probe attached to a solid support (substrate) to detect the presence of the nucleic acid sequence.

As used herein, the term “substrate”, when used in reference to a substance, structure, surface or material, means a composition comprising a nonbiological, synthetic, nonliving, planar or round surface that is not heretofore known to comprise a specific binding, hybridization or catalytic recognition site or a plurality of different recognition sites or a number of different recognition sites which exceeds the number of different molecular species comprising the surface, structure or material. Examples of the substrate include, but are not limited to, semiconductors, synthetic (organic) metals, synthetic semiconductors, insulators and dopants; metals, alloys, elements, compounds and minerals; synthetic, cleaved, etched, lithographed, printed, machined and microfabricated slides, devices, structures and surfaces; industrial polymers, plastics, membranes silicon, silicates, glass, metals and ceramics; and wood, paper, cardboard, cotton, wool, cloth, woven and nonwoven fibers, materials and fabrics; and amphibious surfaces.

It is known in the art that several types of membranes have adhesion to nucleic acid sequences. Specific non-limiting examples of these membranes include nitrocellulose or other membranes used for detection of gene expression such as polyvinylchloride, diazotized paper and other commercially available membranes such as GENESCREEN™, ZETAPROBE™ (Biorad) and NYTRAN™. Beads, glass, wafer and metal substrates are also included. Methods for attaching nucleic acids to these objects are well known in the art. Alternatively, screening can be done in a liquid phase.

Hybridization Conditions

In nucleic acid hybridization reactions, the conditions used to achieve a particular level of stringency will vary depending on the nature of the nucleic acids being hybridized. For example, the length, degree of complementarity, nucleotide sequence composition (e.g., GC/AT content), and nucleic acid type (e.g., RNA/DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at room temperature (hybridization conditions); 0.2×SSC/0.1% SDS at room temperature (low stringency conditions); 0.2×SSC/0.1% SDS at 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary depending on the particular hybridization reaction involved, and can be determined empirically. In general, conditions of high stringency are used for the hybridization of the probe of interest.

Label

The probe of interest can be detectably labeled, for example, with a radioisotope, a fluorescent compound, a bioluminescent compound, a chemiluminescent compound, a metal chelator, or an enzyme. Appropriate labeling with such probes is widely known in the art and can be performed by any conventional method.

Kit

The present invention provides a kit useful for the detection of a cellular proliferative disorder in a subject.

### EXAMPLES

Hereinafter, the present invention will be described in further detail with reference to examples. It will be obvious to a person having ordinary skill in the art that these examples are illustrative purposes only and are not to be construed to limit the scope of the present invention.

### Example 1

### Discovery of Cervical Cancer-Specific Methylation Gene ACSS3

In order to screen a biomarker which is methylated specifically in cervical cancer, genomic DNAs were isolated from the scrapes of 10 cervical cancer patients and 10 normal persons using the QIAamp DNA Mini kit (QIAGEN, USA). 500 ng of each of the isolated genomic DNAs was sonicated (Vibra Cell, SONICS), thus constructing about 200-300-bp-genomic DNA fragments.

To obtain only methylated DNA from the genomic DNA, a methyl binding domain (Methyl binding domain; MBD2bt) (Moon et al., American Biotechnology Laboratory, 27(10):23-25, 2009) known to bind to methylated DNA was used. Specifically, 2 μg of 6×His-tagged MBD2bt was pre-incubated with 500 ng of the genomic DNA of E. coli JM110 (No. 2638, Biological Resource Center, Korea Research Institute of Bioscience & Biotechnology), and then bound to Ni-NTA magnetic beads (Qiagen, USA). 500 ng of each of the sonicated genomic DNAs isolated from the normal persons and the cervical cancer patients was allowed to react with the beads in the presence of binding buffer solution (10 mM Tris-HCl (pH 7.5), 50 mM NaCl, 1 mM EDTA, 1 mM DTT, 3 mM MgCl₂, 0.1% Triton-X100, 5% glycerol, 25 mg/ml BSA) at 4° C. for 20 minutes. Then, the beads were washed three times with 500 μL of a binding buffer solution containing 700 mM NaCl, and then methylated DNA bound to the MBD2bt was isolated using the QiaQuick PCR purification kit (Qiagen, USA).

Then, the methylated DNAs bound to the MBD2bt were amplified using a genomic DNA amplification kit (Sigma, USA, Cat. No. WGA2), and 4 μg of the amplified DNAs were labeled using a BioPrime Total Genomic Labeling system I (Invitrogen Corp., USA). That is, normal person-derived DNAs were labeled with Cy3 and cervical cancer patient-derived DNA were labeled with Cy5. The reference DNA was mixed with each of the DNAs of the normal persons and the cervical cancer patients, and then hybridized to 244K human CpG microarrays (Agilent, USA) (see FIG. 1). After the hybridization, the DNA mixture was subjected to a series of washing processes, and then scanned using an Agilent scanner. The calculation of signal values from the microarray images was performed by calculating the relative difference in signal strength between the normal person sample and the cervical cancer patient sample using Feature Extraction program v. 9.5.3.1 (Agilent).

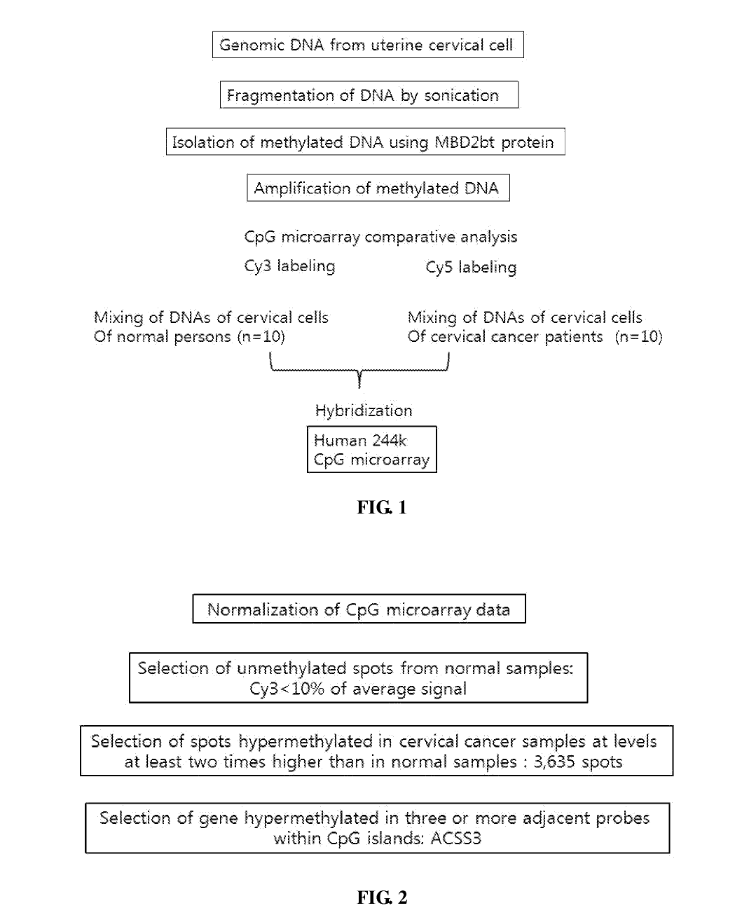

In order to select unmethylated spots from the normal sample, the whole Cy3 signal values were averaged, and then spots having a signal value of less than 10% of the averaged value were regarded as those unmethylated in the samples of the normal persons. From these spots, 3,635 spots were selected, the degree of hypermethylation of which in the cervical cancer samples was at least two times higher than that in the normal samples. In order to select hypermethylated genes having high significance from these spots, analysis was performed, and ACSS3 gene confirmed to be hypermethylated in at least 3 adjacent probes in the CpG islands was selected as a final candidate gene. The presence of CpG islands in the promoter region of the gene was confirmed using MethPrimer (http://itsa.ucsf.edu/˜urolab/methprimer/index1.html), and this gene was secured as a methylation biomarker for diagnosis of cervical cancer (see FIG. 2).

### Example 2

### Measurement of Methylation of Biomarker Gene in Cancer Cell Lines

In order to determine the methylation status of the ACSS3 gene, genomic DNA was isolated from each of the cervical cancer cell lines C33A (ATCC HTB-31), HeLa (Korean Cell Line Bank No. 10002), Caski (Korean Cell Line Bank No. 21550) and SiHa (Korean Cell Line Bank No. 30035), and pyrosequencing for each promoter was carried out.

In order to modify unmethylated cytosine to uracil using bisulfite, 200 ng of the genomic DNA of each cervical cancer cell line was treated with bisulfite using the EZ DNA methylation-gold kit (Zymo Research, USA). When the DNA was treated with bisulfite, unmethylated cytosine was modified to uracil, and the methylated cytosine remained without changes. The DNA treated with bisulfite was eluted in 20 μl of sterile distilled water and subjected to pyrosequencing.

PCR and sequencing primers for performing pyrosequencing for the genes were designed using PSQ assay design program (Biotage, USA). The PCR and sequencing primers for measuring the methylation of each gene are shown in Tables 1 below.

TABLE 1
PCR and sequencing primer sequences

| | | | | | Size of |
|---|---|---|---|---|---|
| | | | SEQ | CpG | amplicon |
| Genes | Primers | Sequences (5′->3′) | ID NOS | location | (bp) |
| ACSS3 | Forward | GGTTTTTTTTTAGGAGTATTTGTGTGT | 3 | −239, | 105 bp |
| | Reverse | Biotin- | | −231, | |
| | | AACCCCAACATACACTTCCAACC | 4 | −215, | |
| | Sequencing | GGAGTATTTGTGTGTTGAA | | −213 | |
| | primer | | 5 | | |

^{a} distances (nucleotides) from the transcription start site (+1): the positions of CpG regions on the genomic DNA used in the measurement of methylation

20 ng of the genomic DNA treated with bisulfite was amplified by PCR. In the PCR amplification, a PCR reaction solution (20 ng of the genomic DNA treated with bisulfite, 5 μl of 10×PCR buffer (Enzynomics, Korea), 5 units of Taq polymerase (Enzynomics, Korea), 4 μl of 2.5 mM dNTP (Solgent, Korea), and 2 μl (10 pmole/μl) of PCR primers) was used, and the PCR reaction was performed under the following conditions: predenaturation at 95° C. for 5 min, and then 45 cycles of denaturation at 95° C. for 40 sec, annealing at 60° C. for 45 sec and extension at 72° C. for 40 sec, followed by final extension at 72° C. for 5 min. The amplification of the PCR product was confirmed by electrophoresis on 2.0% agarose gel.

The amplified PCR product was treated with PyroGold reagents (Biotage, USA), and then subjected to pyrosequencing using the PSQ96MA system (Biotage, USA). After the pyrosequencing, the methylation degree of the DNA was measured by calculating the methylation index. The methylation index was calculated by determining the average rate of cytosine binding to each CpG island.

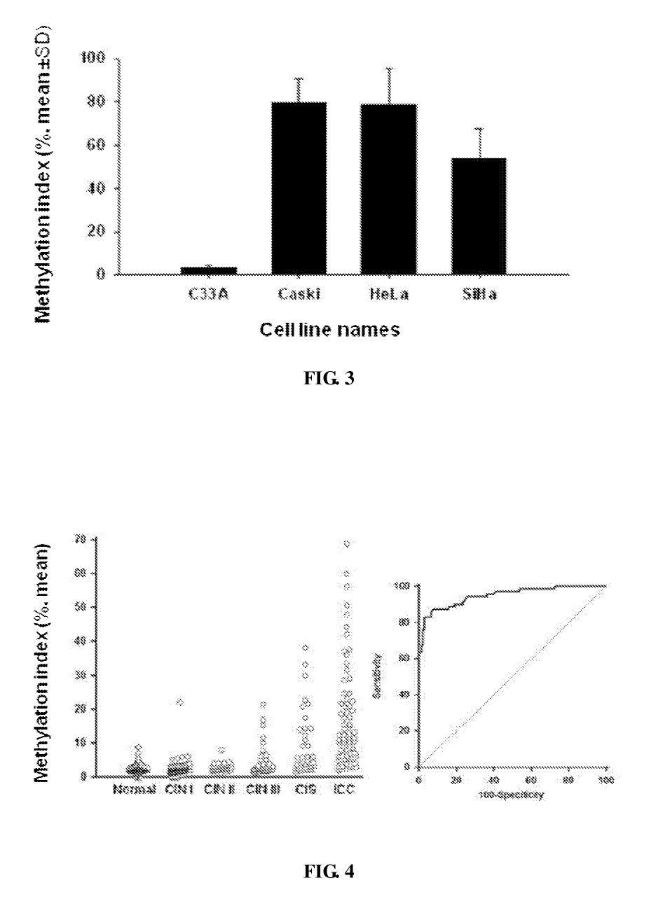

FIG. 3 shows the results of quantitatively measuring the degrees of methylation of the ACSS3 gene biomarker in the cervical cancer cell lines using a pyrosequencing method. As a result, it was shown that the ACSS3 gene biomarker was methylated at a high level in at least one of the cell lines. Table 2 below shows the sequences of the promoters of cervical cancer-specific genes.

TABLE 2
Sequences of methylation marker gene promoters

| Genes | SEQ ID NOS |
|---|---|
| ACSS3 (UTR region sequence) | 1 (2) |
| ADCYAP1 (UTR region sequence) | 6 (7) |
| HOXA11 | 14 |
| VIM | 18 |

### Example 3

### Measurement of Methylation of Biomarker Gene in Scrapes of Cervical Cancer Patients

In order to verify whether the ACSS3 gene can be used as a biomarker for diagnosis of cervical cancer, genomic DNA was isolated from scrape samples of 132 normal persons, 106 CIN I (LSIL) patients, 88 CIN II and CIN III (HSIL) patients, 41 CIS (Carcinoma in situ) patients and 71 cervical cancer patients from the Department of Obstetrics and Gynecology (IRB No. 0904-34), Chungnam National University Hospital, using QIAamp Mini kit (QIAGEN, USA). 200 ng of each of the isolated genomic DNAs was treated with bisulfite using an EZ DNA methylation-Gold kit (Zymo Research, USA). Then, each of the DNAs was eluted in 20 μl of sterile distilled water and subjected to pyrosequencing.

20 ng of the genomic DNA treated with bisulfite was amplified by PCR. In the PCR amplification, a PCR reaction solution (20 ng of the genomic DNA treated with bisulfite, 5 μl of 10×PCR buffer (Enzynomics, Korea), 5 units of Taq polymerase (Enzynomics, Korea), 4 μl of 2.5 mM dNTP (Solgent, Korea), and 2 μl (10 pmole/μl) of PCR primers) was used, and the PCR reaction was performed under the following conditions: predenaturation at 95° C. for 5 min, and then 45 cycles of denaturation at 95° C. for 40 sec, annealing at 60° C. for 45 sec and extension at 72° C. for 40 sec, followed by final extension at 72° C. for 5 min. The amplification of the PCR product was confirmed by electrophoresis on 2.0% agarose gel.

The amplified PCR product was treated with PyroGold reagents (Biotage, USA), and then subjected to pyrosequencing using the PSQ96MA system (Biotage, USA). After the pyrosequencing, the methylation degree of the DNA was measured by calculating the methylation index thereof. The methylation index was calculated by determining the average rate of cytosine binding to each CpG region. In addition, after the methylation index of DNA in the normal persons and the cervical cancer patients has been measured, a methylation index cut-off value for diagnosis of cervical cancer patients was determined through receiver operating characteristic (ROC) curve analysis.

FIG. 4 shows the results of measuring the methylation of the ACSS3 gene in cervical scrapes. As can be seen in FIG. 4, the methylation degree of the gene was higher in the sample of the cervical cancer patients than in the sample of the normal persons. Also, the methylation of the gene was also higher in the sample of the CIN III (HSIL) patients belonging to a high-risk group. FIG. 4 also shows the results of ROC analysis for determining cut-off values for diagnosis of cervical cancer. Also, the results of ROC analysis for determining cut-off values for diagnosis of cervical cancer are shown in Table 3 below. It was shown that the sensitivity of the ACSS3 gene for diagnosis of cervical cancer was 83.1% and the specificity was 97.0%.

TABLE 3
Results of ROC curve analysis for cervical cancer
diagnosis of ACSS3 biomarker

| Criteria | Values |
|---|---|
| Area under ROC curve (AUC) | 0.949 |
| 95% Confidence Interval | 0.908-0.975 |
| P value | 0.0001 |
| Cut-off^{a} | 4.76% |
| Sensitivity | 83.1% (95% CI 72.3-90.9) |
| Specificity | 97.0% (95% CI 92.4-99.1) |

^{a}methylation index criteria for distinction between normal and cancer samples

Table 4 below shows the frequency of methylation-positive samples for ACSS3 gene among a total of 438 cervical samples. As can be seen in Table 4, the frequency of methylation-positive samples started to increase from the CIN III samples belonging to a high-risk group. Such results suggest that the ACSS3 biomarker gene can be used for diagnosis of not only cervical cancer patients, but also identification of patients at risk of progression to cancer among high-risk group patients.

TABLE 4
Frequency of methylation-positive samples for ACSS3 marker gene
among samples of clinical cervical disease patients

| | Number of methylation-positive samples (%) | | | | | | |
|---|---|---|---|---|---|---|---|
| Genes | Normal | CIN I | CIN II | CIN III | CIS | ICC | P-value |
| ACSS3 | 4/132 | 5/106 | 1/42 | 10/46 | 18/41 | 59/71 | 0.0001 |
| | (3.0) | (4.7) | (2.4) | (21.7) | (43.9) | (83.1) | |

Methylation positive criteria: if the methylation index for diagnosis of cervical cancer obtained through the analysis of the ROC (receiver operating characteristic) curve is higher than the methylation index cut off value, it was determined as being positive. On the contrary, if the methylation index is lower than the methylation index cut off value, it was determined as being negative.

### Example 4

### Effect of Combination of ACSS3 Gene with ADCYAP1, HOXA11 and VIM Biomarker Genes on Increase in the Ability of ACSS3 Gene to Diagnose Scrapes of Cervical Cancer Patients

In order to whether a combination of ACSS3 gene with each of 3 biomarker genes ADCYAP1, HOXA11 and VIM (Korean Patent Registration No. 10-43525) has an improved ability to diagnose cervical cancer and identify high-risk group patients, the following test was performed. Specifically, genomic DNA was isolated from scrape samples of 131 normal persons, 73 CIN I (LSIL) patients, 46 CIN II and CIN III (HSIL) patients, 23 CIS (Carcinoma in situ) patients and 37 cervical cancer patients from the Department of Obstetrics and Gynecology (IRB No. 0904-34), Chungnam National University Hospital, using QIAamp Mini kit (QIAGEN, USA). 200 ng of each of the isolated genomic DNAs was treated with bisulfite using an EZ DNA methylation-gold kit (Zymo Research, USA). Then, the DNA was eluted in 20 μl of sterile distilled water and subjected to pyrosequencing using the PCR and sequencing primers shown in Table 5 below.

TABLE 5
Sequences of PCR and sequencing primers

| | | | SEQ | | |
|---|---|---|---|---|---|
| | | | ID | CpG | Size of |
| Genes | Primers | Sequences (5′->3′) | NOS | location | amplicon |
| ADCYAP1^{a} | Forward | GGGTGGATTTAYGGTATTTTGT | 8 | +1, | 345 bp |
| | Reverse | Biotin- | 9 | +8, +11, | |
| | | GGGTAAGGGAGGGAAAATTT | | +21 | |
| | Sequencing | GGTTTTTGTTTAGATATTA | 10 | | |
| | primer | | | | |
| ADCYAP1^{b} | Forward | GGGTTTGGTTAGTTATTGGG | 11 | +305, | 175 bp |
| | Reverse | Biotin- | 12 | +307, | |
| | | TAGAGTTTTTTGGGGTTGGAGGG | | +312, | |
| | Sequencing | AGTAAGTAAGAAGTGGTAGG | 13 | +319 | |
| | primer | | | | |
| HOXA11 | Forward | AGTAAGTTTATGGGAGGGGGATT | 15 | −413, | 243 bp |
| | Reverse | Biotin- | 16 | −403, | |
| | | CCCCCATACAACATACTTATACTC | | −396 | |
| | Sequencing | TAGTTTAGGGTATTTTTTATTTAT | 17 | | |
| | primer | | | | |
| VIM | Forward | GTTTTTGTTTTTTATAGGGTT | 19 | −865, | 118 bp |
| | Reverse | Biotin- | 20 | −863, | |
| | | AACCAAATTAATTCAAATCTCAAC | | −861, | |
| | Sequencing | GGTAGTATTGAGAATTAGTA | 21 | −859 | |
| | primer | | | | |

^{a, b}ADCYAP1 gene pyrosequenced for two regions Y = C or T
^{a}distances (nucleotides) from the transcription start

20 ng of the genomic DNA treated with bisulfite was amplified by PCR. In the PCR amplification, a PCR reaction solution (20 ng of the genomic DNA treated with bisulfite, 5 μl of 10×PCR buffer (Enzynomics, Korea), 5 units of Tag polymerase (Enzynomics, Korea), 4 μl of 2.5 mM dNTP (Solgent, Korea), and 2 μl (10 pmole/μl) of PCR primers) was used, and the PCR reaction was performed under the following conditions: predenaturation at 95° C. for 5 min, and then 45 cycles of denaturation at 95° C. for 40 sec, annealing at 60° C. for 45 sec and extension at 72° C. for 40 sec, followed by final extension at 72° C. for 5 min. The amplification of the PCR product was confirmed by electrophoresis on 2.0% agarose gel.

The amplified PCR product was treated with PyroGold reagents (Biotage, USA), and then subjected to pyrosequencing using the PSQ96MA system (Biotage, USA). After the pyrosequencing, the methylation degree of the DNA was measured by calculating the methylation index. The methylation index was calculated by determining the average rate of cytosine binding to each CpG island. In addition, after the methylation index of DNA in the normal persons and the cervical cancer patients has been measured, a methylation index cut-off value for diagnosis of cervical cancer patients was determined through receiver operating characteristic (ROC) curve analysis.

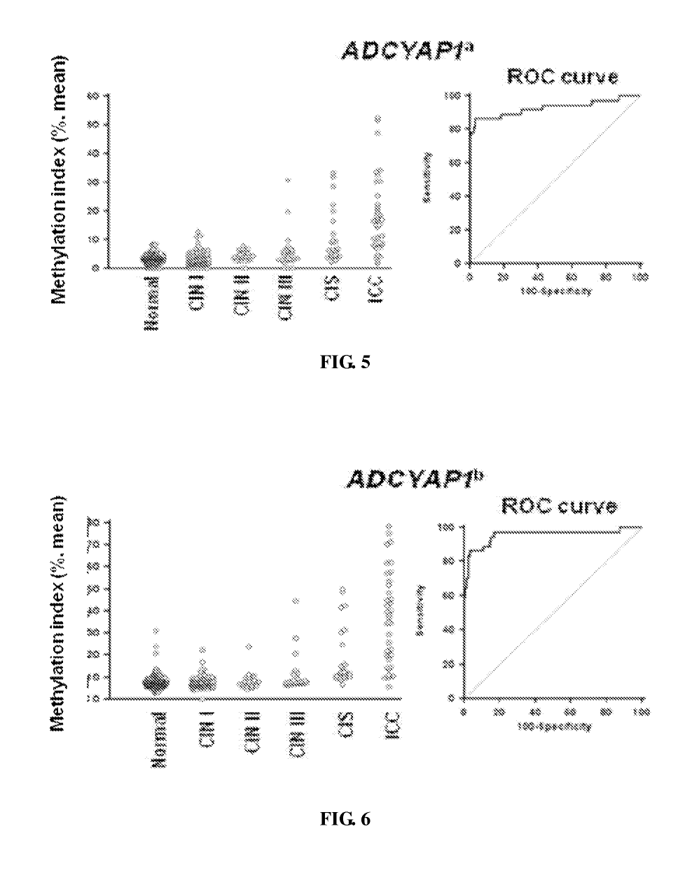

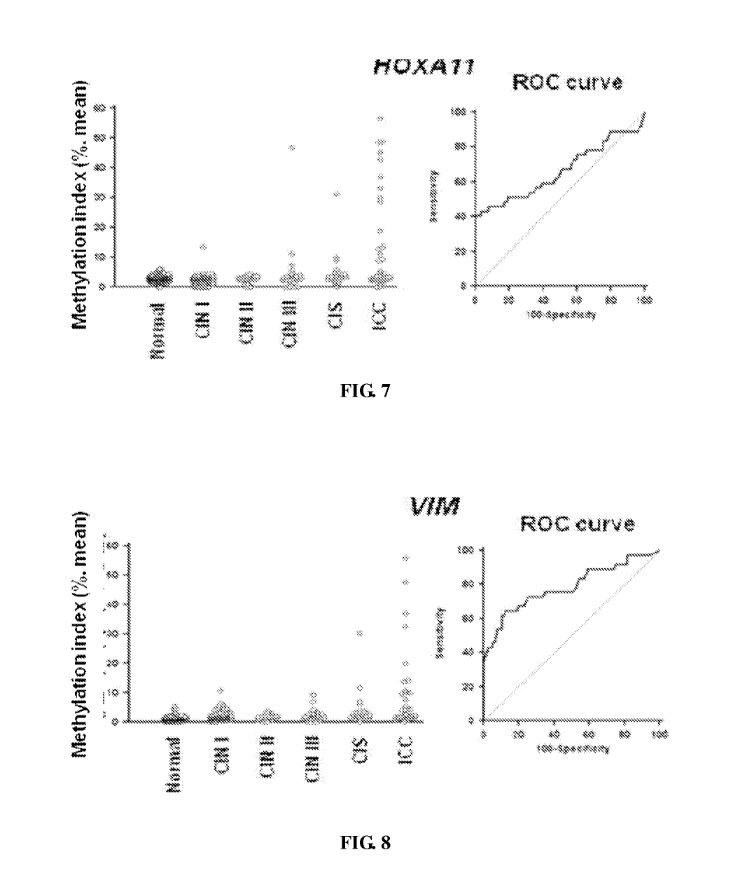

FIGS. 5 to 8 shows the methylation levels of the three biomarker genes in the samples of cervical disease patients and also shows an ROC curve. As can be seen in FIGS. 5 to 8, the 3 biomarker genes were all hypermethylated at high levels in the cervical cancer samples and also hypermethylated at high levels in the high-risk group patients. In addition, the results of ROC curve analysis of each biomarker gene for diagnosis of cervical cancer are shown in Table 6 below.

TABLE 6
Frequencies of methylation-positive samples for ADCYAP1,
HOXA11 and VIM marker genes among samples of clinical
cervical disease patients

| Parameter | ADCYAP1^{a} | ADCYAP1^{b} | HOXA11 | VIM |
|---|---|---|---|---|
| Area under | 0.930 | 0.956 | 0.664 | 0.788 |
| ROC curve | | | | |
| (AUC) | | | | |
| 95% | 0.880-0.963 | 0.913-0.981 | 0.588-0.735 | 0.718-0.847 |
| Confidence | | | | |
| Interval | | | | |
| P value | 0.0001 | 0.0001 | 0.0021 | 0.0001 |
| Cut-off^{c} | 6.48 | 12.83 | 4.32 | 2.09 |
| Sensitivity | 86.5 | 86.5 | 46.0 | 54.1 |
| Specificity | 97.0 | 97.0 | 92.4 | 92.4 |

^{a},^{b}ADCYAP1 gene pyrosequenced for 2 regions
^{c}methylation index criteria for distinction between normal and cancer samples

Also, whether a combination of the ACSS3 gene with each of the 3 biomarker genes has an increased ability to diagnose cervical cancer and to identify high-risk group patients was evaluated.

Table 7 below shows the results of measuring the frequency of methylation-positive samples for a combination of ACSS3 with each of the genes among samples of cervical disease patients. As can be seen in Table 7, a combination of the ACSS3 gene with the ADCYAP1 or VIM gene has an increased sensitivity to the high-risk group CIN II and CIN III and cervical cancer. The use of the ACSS3 gene alone showed a sensitivity of 83.8% to cervical cancer and a sensitivity of only 15.2% to CIN II and CIN III (HSIL; high grade squamous intraepithelial lesion), whereas a combination of the ACSS3 gene with the ADCYAP1 gene showed an increased sensitivity of 91.9-94.6% to cervical cancer and an increased sensitivity of 17.4-26.1% to HSIL. In addition, it was shown that a combination of the ACSS3 gene with the VIM gene showed an insignificantly increased sensitivity to cervical cancer, but the sensitivity thereof to HSIL increased to 32.6%.

TABLE 7
Frequency of methylation-positive samples for combination of ACSS3 gene with each
of ADCYAP1, HOXA11 and VIM marker genes among samples of clinical cervical
disease patients

| | Number of methylation-positive | | | | | | |
|---|---|---|---|---|---|---|---|
| | samples/number of tested samples (%) | | | | | | |
| Genes | Normal | CIN I | CIN II | CIN III | CIS | ICC | P-value |
| ACSS3 | 4/131 | 3/73 | 1/21 | 6/25 | 13/23 | 31/37 | 0.0001 |
| | (3.1) | (4.1) | (4.8) | (24.0) | (56.5) | (83.8) | |
| ACSS3 | 8/131 | 9/73 | 4/21 | 8/25 | 14/23 | 34/37 | 0.0001 |
| or ADCYAP1^{a} | (6.1) | (12.3) | (19.1) | (32.0) | (60.9) | (91.9) | |
| ACSS3 | 7/131 | 6/73 | 2/21 | 6/25 | 14/23 | 35/37 | 0.0001 |
| or ADCYAP1^{b} | (5.3) | (8.2) | (9.5) | (24.0) | (60.9) | (94.6) | |
| ACSS3 or HOXA11 | 7/131 | 5/73 | 1/21 | 8/25 | 15/23 | 32/37 | 0.0001 |
| | (5.3) | (6.8) | (4.8) | (32.0) | (65.2) | (86.5) | |
| ACSS3 or VIM | 14/131 | 4/73 | 4/21 | 11/25 | 18/23 | 32/37 | 0.0001 |
| | (10.7) | (5.5) | (19.0) | (44.0) | (78.3) | (86.5) | |

^{a},^{b}ADCYAP1 pyrosequenced for 2 regions

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

The use of a cervical cancer-specific biomarker according to the present invention, a kit or nucleic acid chip comprising the same, and a method of detecting methylation of the biomarker enables diagnosis of cervical cancer at an initial transformation stage, thus making it possible to diagnosis cervical cancer at an early stage. In addition, the present invention makes it possible to effectively diagnose cervical cancer in a more accurate and rapid manner compared to conventional methods.

1. A composition for diagnosis of cervical cancer, which contains either the promoter CpG island or the UTR CpG island of ACSS3 (NM_{—}024506, acyl-CoA synthetase short-chain family member 3) gene.

2. The composition of claim 1, wherein the CpG island is located at the promoter of the ACSS3 gene comprising a nucleotide sequence of SEQ ID NO: 1 or at the UTR region of the ACSS3 gene comprising a nucleotide sequence of SEQ ID NO: 2.

3. The composition of claim 1, wherein the said composition further contains a CpG island of one gene region selected from the group consisting of the promoter or UTR region of ADCYAP1 (NM-001099733, adenylate cyclase activating polypeptide 1) gene, the promoter of HOXA11 (NM_{—}005523, homeobox A11) gene, and the promoter of VIM (NM_{—}003380, Vimentin) gene.

4. The composition of claim 3, wherein the CpG island is located at the promoter of the ADCYAP1 gene comprising a nucleotide sequence of SEQ ID NO: 1 or at the UTR region of the ADCYAP1 gene comprising a nucleotide sequence of SEQ ID NO: 2.

5. The composition of claim 3, wherein the CpG island is located at the promoter of the HOXA11 gene comprising a nucleotide sequence of SEQ ID NO: 14.

6. The composition of claim 3, wherein the CpG island is located at the promoter of the VIM gene comprising a nucleotide sequence of SEQ ID NO: 18.

7. A kit for diagnosis of cervical cancer, which contains: a PCR primer pair for amplifying a fragment comprising either the promoter CpG island or the UTR CpG island of ACSS3 gene; and a sequencing primer for sequencing a PCR product amplified by the primer pair.

8. The kit of claim 7, wherein the PCR primer pair is a primer pair of SEQ ID NOS: 3 and 4.

9. The kit of claim 7, wherein the sequencing primer is a sequencing primer of SEQ ID No: 5.

10. The kit of claim 7, wherein the said kit further comprises a PCR primer pair for amplifying a fragment comprising the CpG island of one gene region selected from the group consisting of the promoter or UTR region of ADCYAP1 gene, the promoter of HOXA11 gene, and the promoter of VIM gene; and a sequencing primer for sequencing a PCR product amplified by the primer pair.

11. The kit of claim 10, wherein the PCR primer pair is a primer pair selected from the group consisting of a primer pair of SEQ ID NOS: 8 and 9, a primer pair of SEQ ID NOS: 11 and 12, a primer pair of SEQ ID NOS: 15 and 16, and a primer pair of SEQ ID NOS: 19 and 20.

12. The kit of claim 10, wherein the sequencing primer is a sequencing primer selected from the group consisting of a sequencing primer of SEQ ID NO: 10, a sequencing primer of SEQ ID NO: 13, a sequencing primer of SEQ ID NO: 17, and a sequencing primer of SEQ ID NO: 21.

13. A nucleic acid chip for diagnosis of cervical cancer, which comprises a probe capable of hybridizing with a fragment, comprising the promoter CpG island or the UTR CpG island of ACSS3 gene, under strict conditions.

14. The nucleic acid chip of claim 13 for diagnosis of cervical cancer, which comprises a probe capable of hybridizing with a fragment comprising the CpG island of one gene region selected from the group consisting of the promoter or UTR of ADCYAP1 gene, the promoter of HOXA11 gene, and the promoter of VIM gene, under strict conditions.

15. A method of detecting methylation of a cervical cancer-specific biomarker ACSS3 gene, for diagnosis of cervical cancer, the method comprising the steps of:
(a) Preparing a clinical sample containing DNA; and
(b) detecting the methylation of either the promoter CpG island of ACSS3 gene or the UTR CpG island of the ACSS3 gene in the DNA of the clinical sample.

16. The method of claim 15, wherein step (b) comprises a step of determining whether the promoter was methylated by amplifying the isolated DNA using primers which can amplify a fragment comprising the promoter CpG island of ACSS3 gene or the UTR CpG island of the ACSS3 gene, and determining whether an amplification product was obtained from the DNA or whether the nucleotide sequence of the DNA was changed.

17. The method of claim 15, wherein step (b) comprises a step of further detecting methylation of the CpG island of one gene region selected from the group consisting of the promoter or UTR of ADCYAP1 gene, the promoter of HOXA11 gene, and the promoter of VIM gene.

18. The method of claim 15, wherein the methylation detection method is performed using a method selected from the group consisting of PCR, methylation-specific PCR, real-time methylation-specific PCR, PCR using methylated DNA-specific binding protein, quantitative PCR, DNA chip, pyrosequencing, and bisulfite sequencing.

19. The method of claim 15, wherein the clinical sample is selected from the group consisting of tissue, cell, blood, plasma, and urine derived from a patient suspected of cancer or a subject to be diagnosed.

20. A biomarker for diagnosis of cervical cancer, which contains the promoter CpG island or the UTR CpG island of a cervical cancer-specific biomarker ACSS3 (NM_{—}024506, acyl-CoA synthetase short-chain family member 3) gene.


SEQUENCE LISTING (21 sequences)

SEQ ID NO: 1 - DNA, 499 bp, Homo sapiens
cccggagcagtccttgccctggagctgcttgtgtttttagtaaccagggactgagctgattttgaaaaatcaatcaatcaataaataacaatcaaagagccttgcctcaacgcaccgagttgctggtttcctgcaggtcagaggcgatgccacctccaccaacgaccttccaaagattaaaccagccgtgtacaaactgctggattgaaaattgagagatgggccgttggcctcctcctaggagtacttgtgtgttgaaatcggaaggacgttctggagtttgagcgcgatttttcccggaaggctgcggggttggaagtgcatgttggggtttggatctcgtgcagcagcgcaactaacctgctgcaacggcgctgtgacacccctgtcccggggcccccactttagcctgttgcttctctggtcgctccaggtcggtttttcccggcgactctagtgtggcttccaaacttctgggctctgggctcacttcggaatt

SEQ ID NO: 2 - DNA, 91 bp, Homo sapiens
tgcccccgccccctactcccttccctcaggccccaggaagttgcaagagtaccatttgtcgcacactcggggaccgcgggtggccggagga

SEQ ID NO: 3 - DNA, 27 bp, Artificial Sequence
  primer
ggtttttttttaggagtatttgtgtgt

SEQ ID NO: 4 - DNA, 23 bp, Artificial Sequence
  primer
aaccccaacatacacttccaacc

SEQ ID NO: 5 - DNA, 19 bp, Artificial Sequence
  primer
ggagtatttgtgtgttgaa

SEQ ID NO: 6 - DNA, 1000 bp, Homo sapiens
atgttgacaaagagggctctccaaaaaccatgttcggatagatttttgcgaactgcacagataaataggagcagaaggccggtcacctctgtaaccagcggtagcagcagcagaagccgcagcttcagaggcagccggagagacctcggagcagagaaggcgccgccgaccctcgcggctgcctggcccgcggctcctacaaaggcgggctagccgcccgccctctcccttgccttcctccccttcttttctgactttccctctttcccttaatcgcctgcttcttcctccgggtggacttacggccaccttgctcctccgcgcttcacctcatcgccccctctttctttcttctgcctctctctctgcgcccccttctctccgtgtcacgctccctcctggttctgcgcgtctacaaacttttgagcagaacacgagcctcggcaaacgagtcccgcagctcctcctgctgctcccgctggttcctgcggcttctgctcagacaccaacgccagacggcgatgcctctcgggtggtgactccagcgcaggaacttgaagaagcgctttgcccgccgtcctacctggcagctctcctggcagcgggaggagttgaagggtaagggagggaaaatcttaccaaagcgaccggctcactcgactgctgattctttcgcttggcatcgcgtcaggggagttagctttccttcagccgggtctggctagttattgggcgccgggtagatgcatatatatatatttttttctaactatagcaagcaagaagtggcagggcgcgcaccggctgtcgccaagtgctgttcaactcagggagccggggcttcgctccgtccctcccccggcttccagagctttttggggttggagggtgggaggccaggggcgttctcacagctgtgtgtcctctttcccatcctgcgcagaatgaccatgtgtagcggagcgaggctggccctgctggtctatgggataatcatgca

SEQ ID NO: 7 - DNA, 444 bp, Homo sapiens
agacaccaacgccagacggcgatgcctctcgggtggtgactccagcgcaggaacttgaagaagcgctttgcccgccgtcctacctggcagctctcctggcagcgggaggagttgaagggtaagggagggaaaatcttaccaaagcgaccggctcactcgactgctgattctttcgcttggcatcgcgtcaggggagttagctttccttcagccgggtctggctagttattgggcgccgggtagatgcatatatatatatttttttctaactatagcaagcaagaagtggcagggcgcgcaccggctgtcgccaagtgctgttcaactcagggagccggggcttcgctccgtccctcccccggcttccagagctttttggggttggagggtgggaggccaggggcgttctcacagctgtgtgtcctctttcccatcctgcgcaga

SEQ ID NO: 8 - DNA, 22 bp, Artificial Sequence
  primer
gggtggatttayggtattttgt

SEQ ID NO: 9 - DNA, 20 bp, Artificial Sequence
  primer
gggtaagggagggaaaattt

SEQ ID NO: 10 - DNA, 19 bp, Artificial Sequence
  primer
ggtttttgtttagatatta

SEQ ID NO: 11 - DNA, 20 bp, Artificial Sequence
  primer
gggtttggttagttattggg

SEQ ID NO: 12 - DNA, 23 bp, Artificial Sequence
  primer
tagagttttttggggttggaggg

SEQ ID NO: 13 - DNA, 20 bp, Artificial Sequence
  primer
agtaagtaagaagtggtagg

SEQ ID NO: 14 - DNA, 800 bp, Homo sapiens
ggatcccgggtaagacgaaggcccttccgggccctgcggatcagtgacaaaccgcggggagaagccgttctggctgttggcggtttagggacggaaggcactaaagcgcttcggaagtgaccatgaatgagagagtgtaatcaagtcaccgtgcaaatcggacaagccaccaggcaggcacatccacggcttcaaactctggccccgaaggggttccggctagggtcggaggcagaggcgcttcccagagcaagtctatgggagggggactgcgaagaagggggtgcaaatgcgagactccaggagaacagactccgagaccacaggccacacagcgacggactcccacctggctatccccagtccagggcatccctcacccacccggggagctgcgggtgggaggtggggacgagagttgagctctcaccgccctctgcacactcgagaacgaggaccctgcaattgagcacaagcatgctgcatgggggcgcaccccagcctctccgcgcgcgccgggaggccccccagccaacatgagttacaccggcgattacgtgctttcggtgagaacaccgagtgacgatctgttgcttcccctgaggtggctacaaagaaaggaagccgggagggaggggagaggaggaaaaaaaaaaaaggaaagggggggggaaaaggcccggactagctagcagcttgtcaatttcaacatcgggtcacatgaccagcacctccctgctaaggatggggatagatttccacgtcagcttacgtctccaaatttctacttcacggatccg

SEQ ID NO: 15 - DNA, 23 bp, Artificial Sequence
  primer
agtaagtttatgggagggggatt

SEQ ID NO: 16 - DNA, 25 bp, Artificial Sequence
  primer
cccccatacaacatacttatactca

SEQ ID NO: 17 - DNA, 24 bp, Artificial Sequence
  primer
tagtttagggtattttttatttat

SEQ ID NO: 18 - DNA, 700 bp, Homo sapiens
tttcctctttcacccagaattttcagatcttgtttaaaaagttgggtgtggtttcatggggggagggggaagagcgagaggagaccagagggacgggggcggggactctgcaagaaaaaccttcccggtgcaatcgtgatctgggaggcccacgtatggcgcctctccaaaggctgcagaagtttcttgctaacaaaaagtccgcacattcgagcaaagacaggctttagcgagttattaaaaacttaggggcgctcttgtcccccacagggcccgaccgcacacagcaaggcgatggcccagctgtaagttggtagcactgagaactagcagcgcgcgcggagcccgctgagacttgaatcaatctggtctaacggtttcccctaaaccgctaggagccctcaatcggcgggacagcagggcgcggtgagtcaccgccggtgactaagcgaccccacccctctccctcgggctttcctctgccaccgccgtctcgcaactcccgccgtccgaagctggactgagcccgttaggtccctcgacagaacctcccctccccccaacatctctccgccaaggcaagtcgatggacagaggcgcgggccggagcagcccccctttccaagcgggcggcgcgcgaggctgcggcgaggcctgagccctgcgttcctgcgctgtgcgcgcccccaccccgcgttcc

SEQ ID NO: 19 - DNA, 21 bp, Artificial Sequence
  primer
gtttttgttttttatagggtt

SEQ ID NO: 20 - DNA, 24 bp, Artificial Sequence
  primer
aaccaaattaattcaaatctcaac

SEQ ID NO: 21 - DNA, 20 bp, Artificial Sequence
  primer
ggtagtattgagaattagta